(19) United States
(12) Patent Application Publication
(10) Pub. No.: US 2026/0022965 A1
(43) Pub. Date: Jan. 22, 2026

(54) LIGHT INTENSITY MEASURING DEVICE FOR ULTRAVIOLET DISINFECTION CHAMBER AND RELATED METHOD

(71) Applicant: LUMICARE MEDTECH CO., LTD., Shenzhen (CN)

(72) Inventors: Tong LIU, Shenzhen (CN); Ganning CAI, Shenzhen (CN); Weixin LONG, Shenzhen (CN); Bo SUN, Shenzhen (CN); Lihua GUO, Shenzhen (CN)

(73) Assignee: LUMICARE MEDTECH CO., LTD., Shenzhen (CN)

(21) Appl. No.: 19/342,746

(22) Filed: Sep. 29, 2025

Related U.S. Application Data
(63) Continuation of application No. PCT/CN2024/092631, filed on May 11, 2024.

(30) Foreign Application Priority Data
May 19, 2023 (CN) 202310570617.6

(51) Int. Cl. G01J 1/42; A61L 2/10; A61L 2/26; G01J 1/44; G01K 3/00

(52) U.S. Cl. CPC G01J 1/429; A61L 2/10; A61L 2/26; G01J 1/44; G01K 3/005; A61L 2202/11; A61L 2202/122; A61L 2202/123; A61L 2202/14; G01J 2001/444

(57) ABSTRACT

A light intensity measuring device for an ultraviolet disinfection chamber and a related method are provided. The light intensity measuring device for the ultraviolet disinfection chamber includes a base, a photocell array rotatably arranged at the base and configured to simultaneously measure the ultraviolet light intensity in the ultraviolet disinfection chamber at multiple points, and a supporting component arranged at the chamber top of the photocell array.

### CROSS-REFERENCE TO RELATED APPLICATIONS

This application is a continuation application of International Application No. PCT/CN2024/092631, filed on May 11, 2024, which claims priority to Chinese Patent Application No. 202310570617.6, filed on May 19, 2023. The disclosures of the above-mentioned applications are incorporated herein by reference in their entireties.

### TECHNICAL FIELD

The present application relates to the technical field of light intensity calibration, and in particular to a light intensity measuring device for an ultraviolet disinfection chamber and a related method.

### BACKGROUND

The surface of the ultrasound probe is a smooth curved surface, and any part of the curved surface needs to be irradiated with ultraviolet C (UVC) light of sufficient intensity. The ultraviolet C disinfector (UVC-DS) is an instrument that performs high-level disinfection on the entire surface of the ultrasound probe. The disinfection principle is to put the probe into the disinfection chamber, and the surfaces of the disinfection chamber (including the bottom surface) surround the probe and are evenly distributed with ultraviolet C light-emitting diode (UVC LED) arrays. During disinfection, all UVC LEDs are lit, so that a uniform UVC light intensity distribution is formed on the probe surface. As the disinfection time increases, the UVC light dose increases, so that the probe surface reaches a high level of disinfection.

During the production inspection and use of UVC-DS, it is necessary to detect the internal UVC light intensity to determine whether the light intensity at each position meets the light intensity requirements and whether it meets the use requirements of high-level disinfection. Since the surface disinfection of the probe is carried out on the entire envelope surface of the probe, and the irradiation of each UVC LED can only cover a small patch area, the internal UVC light intensity cannot be detected only at individual positions, but the light at different positions should be sampled as much as possible and in all directions. The existing technology lacks a measuring instrument that can be used for the light intensity calibration of ultraviolet disinfection devices (UVC-DS) and can simultaneously measure the UVC light intensity at multiple points in different locations.

Therefore, the related art still needs to be improved and developed.

### SUMMARY

The present application provides a light intensity measuring device for an ultraviolet disinfection chamber and a related method, aiming to solve the technical problems mentioned in the background technology of the related art.

The technical solution of the present application is as follows:

The present application provides a light intensity measuring device for an ultraviolet disinfection chamber, including:
a base;
a photocell array rotatably arranged at the base and configured to simultaneously measure an ultraviolet light intensity in the ultraviolet disinfection chamber at multiple points; and
a supporting component arranged at a top of the photocell array;
the base is placed on a chamber bottom of the ultraviolet disinfection chamber, and the supporting component is configured to clamp to a wire passing notch on a chamber top of the ultraviolet disinfection chamber.

In an embodiment, a rotation angle positioning mechanism is arranged between the photocell array and the base; and
the rotation angle positioning mechanism includes a sliding groove arranged at the base and a sliding bar arranged at the photocell array, and the sliding bar extends into the sliding groove and slides directionally along the sliding groove.

In an embodiment, the light intensity measuring device for an ultraviolet disinfection chamber further includes:
a chamber wall abutting member, the chamber wall abutting member is connected to the base through a connecting rod, and the chamber wall abutting member is configured to support a chamber wall of the ultraviolet disinfection chamber.

In an embodiment, the base is provided with a hemispherical foot on a side for contacting the chamber bottom of the ultraviolet disinfection chamber.

In an embodiment, the photocell array includes a plurality of photocell bar plates and a bracket for fixing the plurality of photocell bar plates, and a plurality of photocells are distributed at the photocell bar plates.

The present application also provides a method for evaluating a light intensity uniformity in an ultraviolet disinfection chamber, including:
fixing a light intensity measuring device in the ultraviolet disinfection chamber, the light intensity measuring device includes a base, a photocell array rotatably arranged at the base and configured to simultaneously measure an ultraviolet light intensity in the ultraviolet disinfection chamber at multiple points, and a supporting component arranged at a top of the photocell array;
starting an ultraviolet light source in the ultraviolet disinfection chamber to start disinfection, and obtaining a temperature in the ultraviolet disinfection chamber;
in response to that the temperature in the ultraviolet disinfection chamber reaches a preset temperature, obtaining an integrating illumination value of each photocell on the photocell array at an initial angle by using a fixed duration integrating illumination algorithm; and
performing statistical analysis on the integrating illumination value of each photocell on the photocell array at the initial angle to evaluate an ultraviolet light intensity uniformity in the ultraviolet disinfection chamber at the initial angle.

In an embodiment, the method for evaluating the light intensity uniformity in the ultraviolet disinfection chamber further includes:
turning off the ultraviolet light source in the ultraviolet disinfection chamber, and rotating the photocell array to a first angle after the ultraviolet disinfection chamber cools down;
restarting the ultraviolet light source in the ultraviolet disinfection chamber again to start disinfection, and obtaining the temperature in the ultraviolet disinfection chamber;
in response to that the temperature in the ultraviolet disinfection chamber reaches the preset temperature, obtaining the integrating illumination value of each photocell on the photocell array at the first angle by using the fixed duration integrating illumination algorithm; and
performing statistical analysis on the integrating illumination value of each photocell on the photocell array at the first angle to evaluate the ultraviolet light intensity uniformity in the ultraviolet disinfection chamber at the first angle.

In an embodiment, the in response to that the temperature in the ultraviolet disinfection chamber reaches the preset temperature, obtaining the integrating illumination value of each photocell on the photocell array at the initial angle by using the fixed duration integrating illumination algorithm includes:
in response to that the temperature in the ultraviolet disinfection chamber reaches the preset temperature, obtaining, by sampling, a light intensity sequence of each photocell on the photocell array at the initial angle at fixed time points separated within a preset fixed time period; and
performing an integral calculation with preset integral calculation on the light intensity sequence of each photocell on the photocell array at the initial angle, and obtaining the integrating illumination value of each photocell on the photocell array at the initial angle;
the performing statistical analysis on the integrating illumination value of each photocell on the photocell array at the initial angle to evaluate the ultraviolet light intensity uniformity in the ultraviolet disinfection chamber at the initial angle includes:
calculating a standard deviation of the integrating illumination value of each photocell on the photocell array at the initial angle; and
obtaining, by evaluating, the ultraviolet light intensity uniformity in the ultraviolet disinfection chamber at the initial angle through a threshold range where the standard deviation of the integrating illumination value of each photocell on the photocell array at the initial angle lies.

In an embodiment, the in response to that the temperature in the ultraviolet disinfection chamber reaches the preset temperature, obtaining the integrating illumination value of each photocell on the photocell array at the first angle by using the fixed duration integrating illumination algorithm includes:
in response to that the temperature in the ultraviolet disinfection chamber reaches the preset temperature, obtaining, by sampling, a light intensity sequence of each photocell on the photocell array at the first angle at fixed time points separated within a preset fixed time period; and
performing an integral calculation with a preset integrating algorithm on the light intensity sequence of each photocell on the photocell array at the first angle, and obtaining the integrating illumination value of each photocell on the photocell array at the first angle;
the performing statistical analysis on the integrating illumination value of each photocell on the photocell array at the first angle to evaluate the ultraviolet light intensity uniformity in the ultraviolet disinfection chamber at the first angle includes:
calculating a standard deviation of the integrating illumination value of each photocell on the photocell array at the first angle; and
obtaining, by evaluating, the ultraviolet light intensity uniformity in the ultraviolet disinfection chamber at the first angle through a threshold range where the standard deviation of the integrating illumination value of each photocell on the photocell array at the first angle lies.

The present application also provides a method for calibrating the light intensity of an ultraviolet disinfection chamber, including:
fixing a light intensity measuring device in the ultraviolet disinfection chamber, the light intensity measuring device includes a base, a photocell array rotatably arranged at the base and configured to simultaneously measure an ultraviolet light intensity in the ultraviolet disinfection chamber at multiple positions, and a supporting component arranged at a top of the photocell array;
rotating the photocell array to a calibration angle;
starting an ultraviolet light source in the ultraviolet disinfection chamber to start disinfection and obtain a temperature in the ultraviolet disinfection chamber;
in response to that the temperature in the ultraviolet disinfection chamber reaches a preset temperature, obtaining an integrating illumination value of each photocell on the photocell array at the calibration angle and the integrating illumination value of multiple photocells built in the ultraviolet disinfection chamber by using a fixed duration integrating illumination algorithm;
calculating a weighted average or an arithmetic average of the integrating illumination values of each photocell on the photocell array at the calibration angle; and
for each photocell built in the ultraviolet disinfection chamber, obtaining a dose coefficient of the photocell in the ultraviolet disinfection chamber that needs to be calibrated by dividing the weighted average or arithmetic average by the integrating illumination value of the photocell in the ultraviolet disinfection chamber.

The present application provides a light intensity measuring device for an ultraviolet disinfection chamber and a related method. The light intensity measuring device for the ultraviolet disinfection chamber includes a base, a photocell array rotatably arranged at the base and configured to simultaneously measure the ultraviolet light intensity in the ultraviolet disinfection chamber at multiple points, and a support component arranged on the top of the photocell array. When the light intensity measuring device of the present application is in use, the light intensity measuring device is placed on the chamber bottom of the ultraviolet disinfection chamber with its base, and the supporting component is clamped to the wire passing notch on the chamber top of the ultraviolet disinfection chamber and fixed in the ultraviolet disinfection chamber. During calibration, the photocell array can collect the ultraviolet light intensity at multiple points in the ultraviolet disinfection chamber, and the photocell array can also be rotated to measure the ultraviolet light intensity at different angles. In this way, the light intensity of the ultraviolet disinfection chamber obtained is more comprehensive, and the final evaluation of the uniformity of the light intensity inside the ultraviolet disinfection chamber can be more accurate.

### BRIEF DESCRIPTION OF THE DRAWINGS

FIG. 1 is a schematic structural diagram of a light intensity measuring device for an ultraviolet disinfection chamber according to an embodiment of the present application.

FIG. 2 is a schematic diagram of the installation of the light intensity measuring device for the ultraviolet disinfection chamber according to an embodiment of the present application.

FIG. 3 is a schematic structural diagram of the connection of a photocell array and a base according to an embodiment of the present application.

FIG. 4 is a flowchart of a method for evaluating a light intensity uniformity in the ultraviolet disinfection chamber according to an embodiment of the present application.

FIG. 5 is a relational diagram of the relative illumination changing with time in the ultraviolet disinfection chamber according to an embodiment of the present application.

FIG. 6 is a relational diagram of the relative illumination changing with temperature in the ultraviolet disinfection chamber according to an embodiment of the present application.

FIG. 7 is a relational diagram of between the measuring value (current) of the photocell and the optical power according to an embodiment of the present application.

FIG. 8 is a flowchart of a method for calibrating the light intensity of the ultraviolet disinfection chamber according to an embodiment of the present application.

### DETAILED DESCRIPTION OF THE EMBODIMENTS

In order to make the technical problems to be solved, technical solutions and beneficial effects of the present application clearer and more understandable, the present application is described in further detail hereinafter in conjunction with the drawings and embodiments. It should be understood that the specific embodiments described herein are only used to explain, but not limit, the present application.

In the description of the present application, it needs to be understood that the orientation or positional relationship indicated by the terms “length”, “width”, “up”, “down”, “front”, “back”, “left”, “right”, “vertical”, “horizontal”, “top”, “bottom”, “inside”, “outside”, etc. are based on the orientation or position relational shown in the figures. It is only for the convenience of describing the present application and simplifying the description and does not indicate or imply that the device or element referred to must have a specific orientation, be constructed and operated in a specific orientation, and therefore cannot be understood as a limitation on the present application.

In addition, the terms “first” and “second” are only used for descriptive purposes and they cannot be understood as indicating or implying relative importance or implicitly indicating the number of technical features indicated. Therefore, a feature defined as “first” or “second” may explicitly or implicitly include one or more of the features. In the description of the present application, the meaning of “multiple” is two or more, unless otherwise expressly and specifically defined.

Reference throughout the specification to “an embodiment” or “embodiment” means that a particular feature, structure, or characteristic described in connection with the embodiment is included in at least one embodiment of the present application. Thus, where the phrases “in an embodiment” or “in some embodiments” appear in various places throughout the specification, not all references are to the same embodiment. Furthermore, in one or more embodiments, the particular features, structures, or characteristics may be combined in any suitable manner.

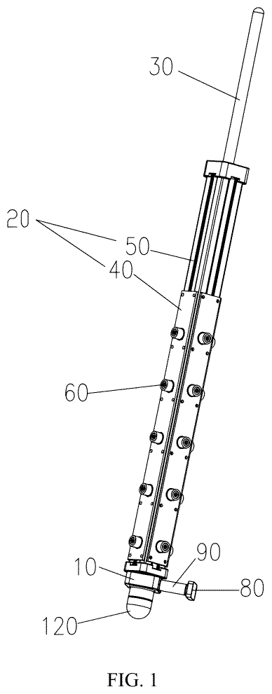

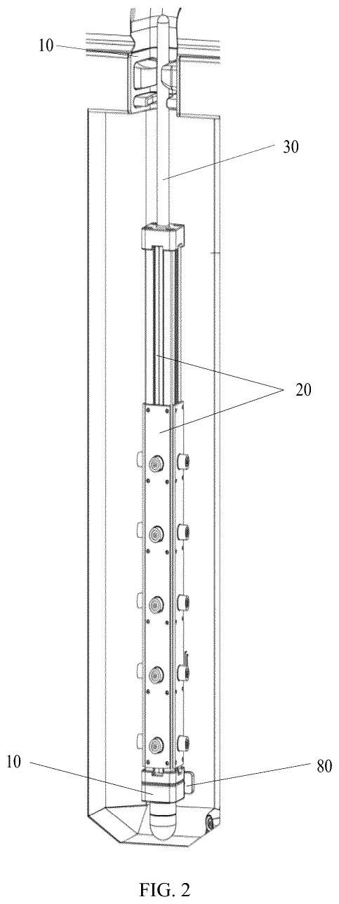

Referring to FIG. 1 and FIG. 2, the first aspect of the present application provides a light intensity measuring device for an ultraviolet disinfection chamber, including a base 10, a photocell array 20 and a supporting component 30. The photocell array 20 is rotatably arranged at the base 10 and is configured to simultaneously measure the ultraviolet light intensity in the ultraviolet disinfection chamber at multiple points. The supporting component 30 (such as a supporting rod) arranged at the top of the photocell array 20. The photocell array 20 includes a plurality of photocell bar plates 40 and a bracket 50 for fixing the plurality of photocell bar plates 40, and the photocell bar plates 40 is provided with a plurality of photocells 60. In an embodiment, in the present application, the bracket 50 is a columnar bracket, and the photocell bar plates 40 are provided with a plurality of photocells 60 in the length direction of the columnar bracket 50. The base 10 is placed on the chamber bottom of the ultraviolet disinfection chamber, and the supporting component 30 is configured to clamp to the wire passing notch 70 on the chamber top of the ultraviolet disinfection chamber. The photocell array 20 in the light intensity measuring device of the present application can be rotated at different angles along the axis to measure the light intensity at various angles in the ultraviolet disinfection chamber.

In an embodiment, the column bracket 50 may be a cylinder or a prism (e.g., a cuboid, a hexagonal prism, etc.). When the column bracket 50 is a cuboid, four photocell bar plates 40 are provided, and the four photocell bar plates 40 are respectively mounted at the four sides of the column bracket 50. On the base 10, the central axis of the column bracket 50 is arranged perpendicular to the base 10, and a plurality of photocells 60 are arranged at intervals along the length direction on each photocell bar plate 40. In terms of the number of the photocells 60, in an embodiment, more than two (e.g., five) photocells 60 are arranged in the length direction of each photocell bar plate 40, and the adjacent photocells 60 are arranged at equal intervals in the length direction of the photocell bar plate 40.

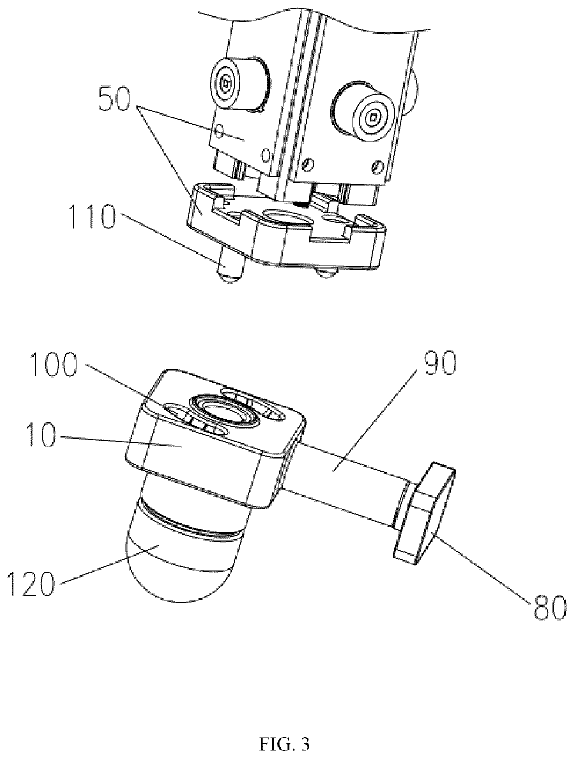

Referring to FIG. 1 and FIG. 3, in an embodiment, the light intensity measuring device of the ultraviolet disinfection chamber further includes a chamber wall abutting member 80. The chamber wall abutting member 80 is connected to the side of the base 10 through a connecting rod 90, and the chamber wall abutting member 80 is configured to support the chamber wall of the ultraviolet disinfection chamber. In the present application, the shape of the chamber wall abutting member 80 can be, for example, a square piece, and the function of the chamber wall abutting member 80 is mainly to facilitate the calibration of the verticality of the light intensity measuring device through the chamber wall, so that the light intensity measuring device is located exactly in the center of the ultraviolet disinfection chamber.

Referring to FIG. 3, in an embodiment, a rotation angle positioning mechanism is arranged between the photocell array 20 and the base 10. The rotation angle positioning mechanism includes a sliding groove 100 arranged at the base 10 and a sliding bar 110 arranged at the photocell array 20, and the sliding bar 110 extends into the sliding groove 100 and slides directionally along the sliding groove 100. In the present application, the sliding groove 100 and the sliding bar 110 can be provided in multiple groups, for example, two groups. The two groups of the sliding grooves 100 and the sliding bar 110 are symmetrical about the center of the base 10, and the two groups of the sliding grooves 100 are both arc-shaped. The sliding bar 110 rotates to the two ends of the sliding groove 100. One state is that the end face of the photocell array 20 coincides with the surface of the base 10 (that is, the edges of the end of the photocell array 20 are parallel to the edges of the base 10), and the other state is that the edges of the photocell array 20 are perpendicular to the diagonal of the base 10.

Referring to FIG. 3, in an embodiment, the base 10 is provided with a hemispherical foot 120 on one side for contacting the chamber bottom of the ultraviolet disinfection chamber. In the present application, the hemispherical foot 120 has two main functions, one of the two main functions is to raise the height of the base 10 and the chamber bottom of the ultraviolet disinfection chamber, and the other of the two main functions is that the base 10 and the chamber bottom of the ultraviolet disinfection chamber are in point contact, which can be conveniently applied to the chamber bottom of more shapes.

The main functions of the light intensity measuring device for the ultraviolet disinfection chamber of the present application are as follows:
(1) During production, when inspecting the product quality, the device can be configured to check whether the UVC light intensity at each position in the disinfection chamber meets the qualified standard requirements.
(2) During production, the disinfection dose monitoring system of the ultraviolet disinfector (UVC-DS) itself is subjected to dose calibration.
(3) During use, the disinfection dose is monitored regularly to promptly discover the light intensity attenuation and unevenness caused by various reasons (such as light intensity attenuation due to lamp bead aging, circuit reasons, etc.).
(4) During use, the disinfection dose monitoring system of the ultraviolet disinfector (UVC-DS) itself is periodically subjected to dose recalibration.

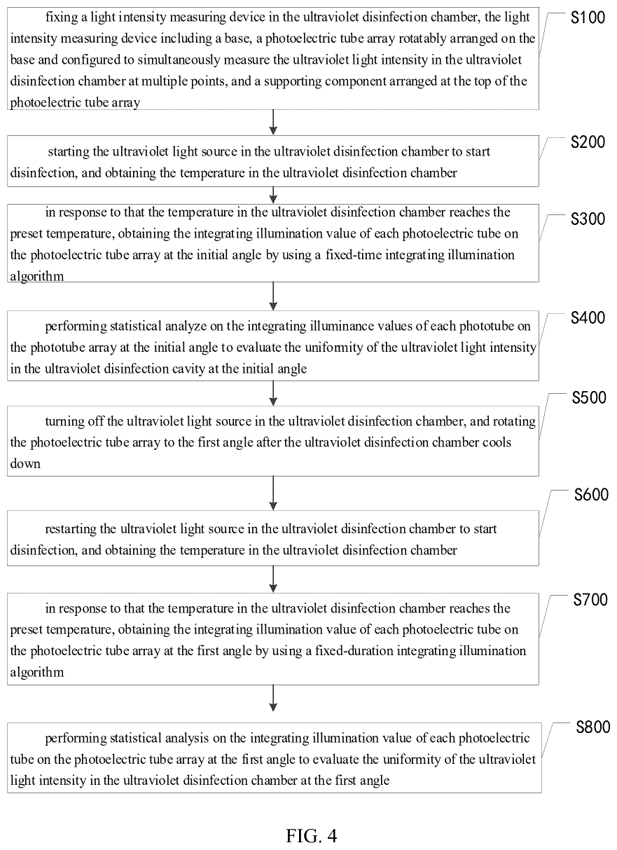

Referring to FIG. 4, the second aspect of the present application provides a method for evaluating a light intensity uniformity in the ultraviolet disinfection chamber, including the following steps:
S100, fixing a light intensity measuring device in the ultraviolet disinfection chamber, the light intensity measuring device including a base, a photocell array rotatably arranged on the base and configured to simultaneously measure the ultraviolet light intensity in the ultraviolet disinfection chamber at multiple points, and a supporting component arranged at the top of the photocell array. In an embodiment, this step is to place the base of the light intensity measuring device on the chamber bottom of the ultraviolet disinfection chamber and clamp the supporting component to the wire passing notch on the chamber top of the ultraviolet disinfection chamber, and the light intensity measuring device is preset to an initial angle state (i.e., the edges at the ends of the photocell array are parallel to the edges of the base).
S200, starting the ultraviolet light source in the ultraviolet disinfection chamber to start disinfection, and obtaining the temperature in the ultraviolet disinfection chamber. In the present application, the method of obtaining the temperature can be the temperature sensor built into the ultraviolet disinfection chamber of the ultraviolet disinfector (UVC-DS), or it can be another selected temperature sensor, and start measuring when the temperature reaches a certain fixed temperature (such as 45° C.) (the advantage of this measuring step is that it avoids errors caused by inconsistent measuring temperature and fluctuations in the illumination of the photocell, and has high accuracy).
S300, in response to that the temperature in the ultraviolet disinfection chamber reaches the preset temperature, obtaining the integrating illumination value of each photocell on the photocell array at the initial angle by using a fixed duration integrating illumination algorithm. In step S300, each photocell reading is integrating for a fixed duration (such as 10 seconds), and the integrating illumination (lighting dose) of the fixed duration (such as 10 seconds) is obtained, and the measuring is stopped.

In an embodiment, in response to that the temperature in the ultraviolet disinfection chamber reaches the preset temperature in step S300, obtaining the integrating illumination value of each photocell on the photocell array at the initial angle by using a fixed duration integrating illumination algorithm, including:
in response to that the temperature in the ultraviolet disinfection chamber reaches the preset temperature, obtaining, by sampling, a light intensity sequence of each photocell on the photocell array at the initial angle at fixed time points separated within a preset fixed time period. For example, the light intensity sequence is sampled at fixed time points separated by a fixed time period (for example, at intervals of 100 ms); and
performing an integral calculation with a preset integrating algorithm on the light intensity sequence of each photocell on the photocell array at the initial angle, and obtaining an integrating illumination value of each photocell on the photocell array at the initial angle.

The preset integrating algorithm includes: rectangular accumulation method (Riemann Sums rule), trapezoidal accumulation method (Trapezoidal Rule), etc.
S400, performing statistical analysis on the integrating illuminance values of each photocell on the photocell array at the initial angle to evaluate the ultraviolet light intensity uniformity in the ultraviolet disinfection chamber at the initial angle. In the present application, the integrating illuminance value of each photocell is read for evaluation, and the evaluation content includes (but is not limited to): if there is a value below the minimum limit, it may be that the disinfection light intensity cannot meet the needs; performing statistical analysis on the readings of the photocell array, such as calculating the standard deviation, can be configured to understand whether the uniformity of the light intensity in the disinfection chamber meets the needs. The reading step can be to connect the cable of the light intensity measuring device to the external reading device for reading, or to connect the cable of the measuring device to the USB port of UVC-DS to allow UVC-DS to read, or to connect the measuring device to a wireless device such as Bluetooth to allow UVC-DS or an external reading device to read.

In an embodiment, the step S400 of performing statistical analysis on the integrating illumination values of each photocell on the photocell array at the initial angle to evaluate the ultraviolet light intensity uniformity in the ultraviolet disinfection chamber at the initial angle includes: calculating the standard deviation of the integrating illumination values of each photocell on the photocell array at the initial angle; obtaining, by evaluating, the ultraviolet light intensity uniformity in the ultraviolet disinfection chamber at the initial angle through a threshold range where the standard deviation of the integrating illumination value of each photocell on the photocell array at the initial angle lies.

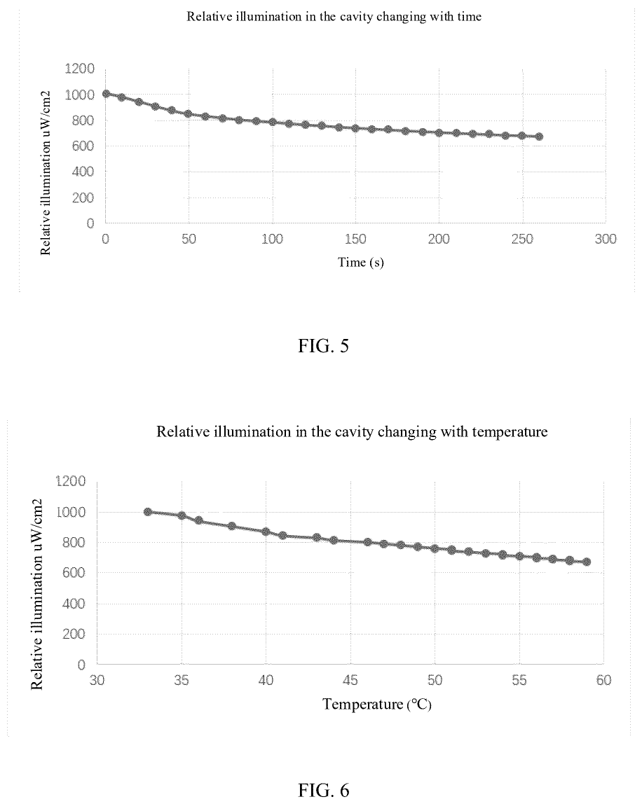

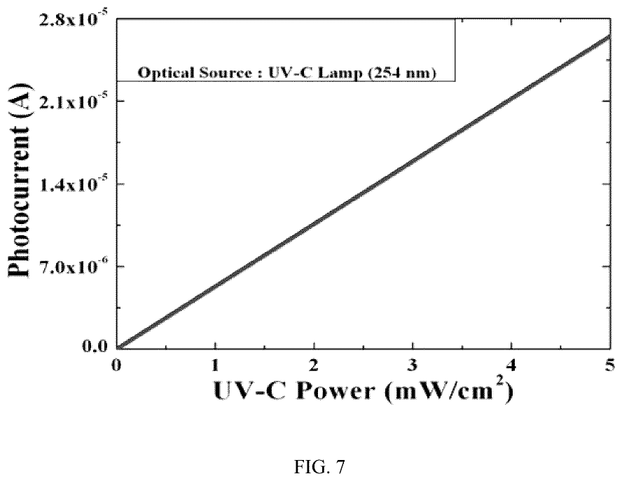

Referring to FIG. 5, FIG. 6 and FIG. 7, when measuring the light intensity inside the ultraviolet disinfection chamber of the ultraviolet disinfector (UVC-DS), the light intensity of the UVC LED fluctuates with various factors. The temperature rise leads to a decrease in light intensity, and the temperature decrease leads to an increase in light intensity. The extended use time leads to a decrease in light intensity. The current fluctuation leads to light intensity fluctuations. If the light intensity is simply measured in real time, the value will fluctuate all the time. It is impossible to accurately measure a value, and it is impossible to judge whether the light intensity meets the requirements. By using the fixed temperature measuring and the integrating illuminance statistical illuminance value method of this application, the evaluation standard consistency is stronger and the evaluation result is more reliable.

In the second aspect of the present application, after the test at one angle is completed, the light intensity measuring device can be rotated to another specified angle to perform light intensity tests at different angles. Referring to FIG. 4, in an embodiment, the light intensity uniformity evaluation method of the ultraviolet disinfection chamber further includes:
S500, turning off the ultraviolet light source in the ultraviolet disinfection chamber, and rotating the photocell array to the first angle after the ultraviolet disinfection chamber cools down;
S600, restarting the ultraviolet light source in the ultraviolet disinfection chamber to start disinfection, and obtaining the temperature in the ultraviolet disinfection chamber;
S700, in response to that the temperature in the ultraviolet disinfection chamber reaches the preset temperature, obtaining the integrating illumination value of each photocell on the photocell array at the first angle by using a fixed duration integrating illumination algorithm; and
S800, performing statistical analysis on the integrating illumination value of each photocell on the photocell array at the first angle to evaluate the ultraviolet light intensity uniformity in the ultraviolet disinfection chamber at the first angle. In this application, the specific implementation of steps S500-S800 is the same as the specific implementation of steps S100-S400, except that the angle of the photocell array in the ultraviolet disinfection chamber is different, and this application will not be repeated in detail again.

In an embodiment, the in response to that the temperature in the ultraviolet disinfection chamber reaches the preset temperature, obtaining the integrating illumination value of each photocell on the photocell array at the first angle by using the fixed duration integrating illumination algorithm includes:
in response to that the temperature in the ultraviolet disinfection chamber reaches the preset temperature, obtaining, by sampling, the light intensity sequence of each photocell on the photocell array at the first angle at fixed time points separated within a preset fixed time period; and
performing an integral calculation with a preset integral calculation on the light intensity sequence of each photocell on the photocell array at the initial angle, and obtaining an integrating illumination value of each photocell on the photocell array at the initial angle;
The performing statistical analysis on the integrating illumination value of each photocell on the photocell array at the initial angle to evaluate the ultraviolet light intensity uniformity in the ultraviolet disinfection chamber at the initial angle includes:
calculating the standard deviation of the integrating illumination value of each photocell on the photocell array at the first angle; and
obtaining, by evaluating, the ultraviolet light intensity uniformity in the ultraviolet disinfection chamber at the first angle through a threshold range where the standard deviation of the integrating illumination value of each photocell on the photocell array at the first angle lies.

In the present application, in addition to measuring the uniformity of the light intensity in the ultraviolet disinfection chamber, the light intensity measuring device can also be configured to calibrate the disinfection dose monitoring system in the ultraviolet disinfection chamber. The disinfection dose monitoring system is a component of the ultraviolet disinfector (UVC-DS). It determines whether the UVC light reaches the dose requirement of high-level disinfection by real-time monitoring of the UVC light dose during the disinfection process, and notifies the disinfection control system to end the disinfection when the dose requirement is reached. The dose monitoring system monitors the UVC light dose in real time through UVC photocells mounted at several (such as two) positions in the disinfection chamber. Before leaving the factory and during use, the dose monitoring system needs to be calibrated regularly to ensure accurate disinfection dose monitoring. By comparing the light intensity measured by the light intensity measuring device of the present application with the measured value of the dose monitoring system of UVC-DS, the internal parameters of the dose monitoring system of UVC-DS can be calibrated. This calibration process can be performed by manual calculation or by automatic calculation by reading the value of the light intensity measuring device through UVC-DS.

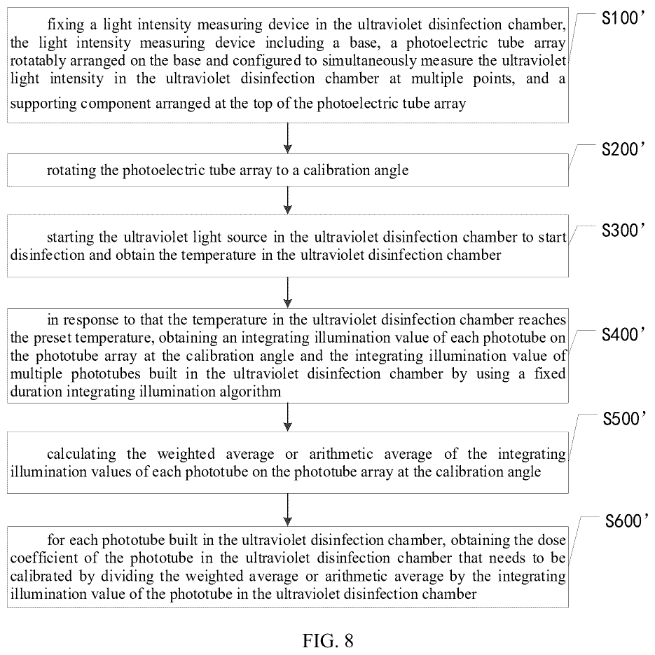

Referring to FIG. 8, the third aspect of the present application provides a method for calibrating the light intensity of an ultraviolet disinfection chamber, including the following steps:
S100′, fixing a light intensity measuring device in the ultraviolet disinfection chamber, the light intensity measuring device including a base, a photocell array rotatably arranged on the base and configured to simultaneously measure the ultraviolet light intensity in the ultraviolet disinfection chamber at multiple points, and a supporting component arranged at the top of the photocell array;
S200′, rotating the photocell array to a calibration angle;
S300′, starting the ultraviolet light source in the ultraviolet disinfection chamber to start disinfection and obtain the temperature in the ultraviolet disinfection chamber;
S400′, in response to that the temperature in the ultraviolet disinfection chamber reaches the preset temperature, obtaining an integrating illumination value of each photocell on the photocell array at the calibration angle and the integrating illumination value of multiple photocells built in the ultraviolet disinfection chamber by using a fixed duration integrating illumination algorithm;
S500′, calculating the weighted average or arithmetic average of the integrating illumination values of each photocell on the photocell array at the calibration angle; and
S600′, for each photocell built in the ultraviolet disinfection chamber, obtaining the dose coefficient of the photocell in the ultraviolet disinfection chamber that needs to be calibrated by dividing the weighted average or arithmetic average by the integrating illumination value of the photocell in the ultraviolet disinfection chamber.

Taking the scenario where the dose monitoring system of the ultraviolet disinfector (UVC-DS) has two UVC photocells as an example, the principle of the light intensity calibration method of this application is explained. When the disinfection starts, the UVC light dose received is calculated in real time. Assume that the reading dose of the two photocells is R₁ and, R₂. The reading of the photocell array of the light intensity measuring device is P₁, P₂, . . . , P_{N}, (here, N is the number of photocells). We calibrate the measured dose of the dose monitoring system according to P₁, P₂, . . . , P_{N}. We let the measured dose of the two photocells in the dose monitoring system be S₁,S₂. Since the light power and the photocell measuring value (current) are in linear proportion, and the reading dose is the fixed duration integrating illumination of the photocell measuring value, the relational between the reading dose value and the measured dose value of the photocell is a linear relational. That is, S₁=a₁·R₁, and, S₂=a₂·R₂, where, a₁ and a₂ are the dose coefficients that need to be calibrated, which can be calibrated by the weighted average or arithmetic mean of P₁, P₂, . . . , P_{N}. That is,

$${S}_{1} = \frac{1}{{\sum }_{l = 1}^{N} {m}_{i}} ( {m}_{1} {P}_{1} + {m}_{2} {P}_{2} + … + {m}_{N} {P}_{N} )$$

$$and$$

$${S}_{2} = \frac{1}{{\sum }_{i = 1}^{N} {n}_{i}} ( {n}_{1} {P}_{1} + {n}_{2} {P}_{2} + … + {n}_{N} {P}_{N} )$$

When m₁=m₂= . . . =mₙ=1, it is the arithmetic mean.

The dose coefficient is as follows:

$${a}_{1} = \frac{{S}_{1}}{{R}_{1}}$$

$$and$$

$${a}_{2} = \frac{{S}_{2}}{{R}_{2}}$$

In general, the light intensity measuring device and related methods of the present application have the following benefits and advantages:
(1) For the ultrasonic probe surface disinfection instrument used for surface disinfection, the UVC light intensity is measured in all directions, at multiple points, and at the same time, and the illuminance at the measuring point is more one-to-one with the actual light intensity received by the ultrasonic probe surface.
(2) Considering that the light intensity of the UVC LED will fluctuate rapidly with time and temperature, the measuring of the contrast is achieved by integrating the illuminance for a fixed duration.
(3) The starting point of the fixed duration integrating illuminance starts at a fixed temperature, which improves the environmental consistency of each measuring (because the temperature of the test environment may be different, and the temperature has a significant effect on the light intensity of the UVC LED).
(4) The light intensity measuring device has a locator in the disinfection chamber, which is configured to uniquely determine the position of the device in the disinfection chamber, so that the measuring results are comparable between different measured UVC-DS and with the control illuminance values.
(5) The multi-point measuring part of the light intensity measuring device can be rotated by a specified angle along the axis to measure different directions.
(6) The UVC-DS's own disinfection dose monitoring system is automatically calibrated.

Although the present application has been disclosed as above by the embodiments, the above embodiments are not intended to limit the present application. Those of ordinary skill in the art can make various changes and modifications without departing from the spirit and scope of the present application. Therefore, the scope of protection of the present application shall be based on the scope defined in the claims.

What is claimed is:

1. A light intensity measuring device for an ultraviolet disinfection chamber, comprising:
a base;
a photocell array rotatably arranged at the base and configured to simultaneously measure an ultraviolet light intensity in the ultraviolet disinfection chamber at multiple points; and
a supporting component arranged at a top of the photocell array;
wherein the base is placed on a chamber bottom of the ultraviolet disinfection chamber, and the supporting component is configured to clamp to a wire passing notch on a chamber top of the ultraviolet disinfection chamber.

2. The light intensity measuring device for the ultraviolet disinfection chamber according to claim 1, wherein a rotation angle positioning mechanism is arranged between the photocell array and the base; and
the rotation angle positioning mechanism comprises a sliding groove arranged at the base and a sliding bar arranged at the photocell array, and the sliding bar extends into the sliding groove and slides directionally along the sliding groove.

3. The light intensity measuring device for the ultraviolet disinfection chamber according to claim 1, further comprising:
a chamber wall abutting member, wherein the chamber wall abutting member is connected to the base through a connecting rod, and the chamber wall abutting member is configured to support a chamber wall of the ultraviolet disinfection chamber.

4. The light intensity measuring device for the ultraviolet disinfection chamber according to claim 1, wherein the base is provided with a hemispherical foot on a side for contacting the chamber bottom of the ultraviolet disinfection chamber.

5. The light intensity measuring device for the ultraviolet disinfection chamber according to claim 1, wherein the photocell array comprises a plurality of photocell bar plates and a bracket for fixing the plurality of photocell bar plates, and a plurality of photocells are distributed at the photocell bar plates.

6. A method for evaluating a light intensity uniformity in an ultraviolet disinfection chamber, comprising:
fixing a light intensity measuring device in the ultraviolet disinfection chamber, wherein the light intensity measuring device comprises a base, a photocell array rotatably arranged at the base and configured to simultaneously measure an ultraviolet light intensity in the ultraviolet disinfection chamber at multiple points, and a supporting component arranged at a top of the photocell array;
starting an ultraviolet light source in the ultraviolet disinfection chamber to start disinfection, and obtaining a temperature in the ultraviolet disinfection chamber;
in response to that the temperature in the ultraviolet disinfection chamber reaches a preset temperature, obtaining an integrating illumination value of each photocell on the photocell array at an initial angle by using a fixed duration integrating illumination algorithm; and
performing statistical analysis on the integrating illumination value of each photocell on the photocell array at the initial angle to evaluate an ultraviolet light intensity uniformity in the ultraviolet disinfection chamber at the initial angle.

7. The method for evaluating the light intensity uniformity of the ultraviolet disinfection chamber according to claim 6, further comprising:
turning off the ultraviolet light source in the ultraviolet disinfection chamber, and rotating the photocell array to a first angle after the ultraviolet disinfection chamber cools down;
restarting the ultraviolet light source in the ultraviolet disinfection chamber again to start disinfection, and obtaining the temperature in the ultraviolet disinfection chamber;
in response to that the temperature in the ultraviolet disinfection chamber reaches the preset temperature, obtaining the integrating illumination value of each photocell on the photocell array at the first angle by using the fixed duration integrating illumination algorithm; and
performing statistical analysis on the integrating illumination value of each photocell on the photocell array at the first angle to evaluate the ultraviolet light intensity uniformity in the ultraviolet disinfection chamber at the first angle.

8. The method for evaluating the light intensity uniformity of the ultraviolet disinfection chamber according to claim 6, wherein the in response to that the temperature in the ultraviolet disinfection chamber reaches the preset temperature, obtaining the integrating illumination value of each photocell on the photocell array at the initial angle by using the fixed duration integrating illumination algorithm comprises:
in response to that the temperature in the ultraviolet disinfection chamber reaches the preset temperature, obtaining, by sampling, a light intensity sequence of each photocell on the photocell array at the initial angle at fixed time points separated within a preset fixed time period; and
performing an integral calculation with preset integral calculation on the light intensity sequence of each photocell on the photocell array at the initial angle, and obtaining the integrating illumination value of each photocell on the photocell array at the initial angle;
wherein the performing statistical analysis on the integrating illumination value of each photocell on the photocell array at the initial angle to evaluate the ultraviolet light intensity uniformity in the ultraviolet disinfection chamber at the initial angle comprises:
calculating a standard deviation of the integrating illumination value of each photocell on the photocell array at the initial angle; and
obtaining, by evaluating, the ultraviolet light intensity uniformity in the ultraviolet disinfection chamber at the initial angle through a threshold range where the standard deviation of the integrating illumination value of each photocell on the photocell array at the initial angle lies.

9. The method for evaluating the light intensity uniformity of the ultraviolet disinfection chamber according to claim 7, wherein the in response to that the temperature in the ultraviolet disinfection chamber reaches the preset temperature, obtaining the integrating illumination value of each photocell on the photocell array at the first angle by using the fixed duration integrating illumination algorithm comprises:
in response to that the temperature in the ultraviolet disinfection chamber reaches the preset temperature, obtaining, by sampling, a light intensity sequence of each photocell on the photocell array at the first angle at fixed time points separated within a preset fixed time period; and
performing an integral calculation with a preset integrating algorithm on the light intensity sequence of each photocell on the photocell array at the first angle, and obtaining the integrating illumination value of each photocell on the photocell array at the first angle;
wherein the performing statistical analysis on the integrating illumination value of each photocell on the photocell array at the first angle to evaluate the ultraviolet light intensity uniformity in the ultraviolet disinfection chamber at the first angle comprises:
calculating a standard deviation of the integrating illumination value of each photocell on the photocell array at the first angle; and
obtaining, by evaluating, the ultraviolet light intensity uniformity in the ultraviolet disinfection chamber at the first angle through a threshold range where the standard deviation of the integrating illumination value of each photocell on the photocell array at the first angle lies.

10. A method for calibrating the light intensity of an ultraviolet disinfection chamber, comprising:
fixing a light intensity measuring device in the ultraviolet disinfection chamber, wherein the light intensity measuring device comprises a base, a photocell array rotatably arranged at the base and configured to simultaneously measure an ultraviolet light intensity in the ultraviolet disinfection chamber at multiple positions, and a supporting component arranged at a top of the photocell array;
rotating the photocell array to a calibration angle;
starting an ultraviolet light source in the ultraviolet disinfection chamber to start disinfection and obtain a temperature in the ultraviolet disinfection chamber;
in response to that the temperature in the ultraviolet disinfection chamber reaches a preset temperature, obtaining an integrating illumination value of each photocell on the photocell array at the calibration angle and the integrating illumination value of multiple photocells built in the ultraviolet disinfection chamber by using a fixed duration integrating illumination algorithm;
calculating a weighted average or an arithmetic average of the integrating illumination values of each photocell on the photocell array at the calibration angle; and
for each photocell built in the ultraviolet disinfection chamber, obtaining a dose coefficient of the photocell in the ultraviolet disinfection chamber that needs to be calibrated by dividing the weighted average or arithmetic average by the integrating illumination value of the photocell in the ultraviolet disinfection chamber.
